(19) United States
(12) Patent Application Publication
(10) Pub. No.: US 2004/0225233 A1
(43) Pub. Date: Nov. 11, 2004

(54) Magnetic guidewires

(76) Inventors: Brian J. Frankowski, Pittsburgh, PA (US); William J. Federspiel, Pittsburgh, PA (US); Joseph F. Golob JR., Pittsburgh, PA (US); Brack G. Hattler, Pittsburgh, PA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/434,555

(22) Filed: May 9, 2003

(51) Int. Cl. A61B 5/00; A61M 25/09

(52) U.S. Cl. 600/585

(57) ABSTRACT

A system for placement of a medical device in a body passage including a first and second guidewire, each configured with magnets on their distal ends. The guidewires are inserted through different access sites of a body passage and blindly connected using the magnets. The first guidewire is inserted into a first insertion site and the second guidewire is inserted into a second insertion site. Once the distal ends of the guidewires are attached, the second guidewire can then be removed from the first insertion site, detached from the first guidewire and attached to a medical device. The distal end of the second guidewire can then be pulled through the second insertion site to guide the medical device into a desired location in the body passage.

### FEDERALLY SPONSORED RESEARCH

The U.S. Government has a paid-up license in this invention and the right in limited circumstances to require the patent owner to license others on reasonable terms as provided for by the terms of Grant No. DAMD17-98-1-8638 awarded by the Department of the Army.

### BACKGROUND OF THE INVENTION

The present invention relates to apparatuses and methods used to facilitate the advancement and placement of medical devices in a body passage.

In the field of vascular intervention, devices known as guidewires may be used to facilitate access to a patient's vascular system. Guidewires serve as a conduit for introducing medical devices such as catheters, cutting devices, fiber optics, and ultrasound probes.

Inserting medical devices into the vascular anatomy may be difficult to perform due to the size and rigidity of the device. Often, insertion involves extensive training and the use of fluoroscopy to ensure proper device placement. Thus, a need has arisen to perform a therapeutic intervention from two ends of an affected site in the vascular system. Pulling medical devices from a distant site will ease the insertion process and facilitate proper device position.

As used herein, the term “proximal” refers to the portion of the device closest to the user (outside the body passage). The term “distal” refers to the portion of the device furthest from the user (inside the body passage).

A known prior art device used for grasping the distal end of a guidewire inserted into a vasculature is a distal loop or snare wire. Such a device is inserted into the body via a wire or probe, and may be used to retrieve broken catheters as well as pull guidewires through the vasculature. To implement, the physician introduces a guidewire at a first access site and advances the guidewire distal end along the vasculature. The snare wire is then introduced at a second access site, positioned over the distal end of the guidewire and tightened to grasp the guidewire distal end. This causes the guidewire distal end to transversely orient itself with respect to the axis of the snare. The guidewire is then pulled through the vasculature and out of the second access site. One problem with the distal loop or snare system is that the transversely oriented guidewire distal end may potentially traumatize the blood vessel walls of the vasculature as it passes through the system of the patient. A further problem is that the snare wire can easily be kinked during engagement of the distal end of the guidewire. This renders the snare ineffective. Yet another problem with the distal loop or snare system is that the procedure for engaging the guidewire distal end is a time consuming process. It requires considerable maneuvering of the snare by manipulating the snare wire's proximal end. Considering that fluoroscopy is needed to visualize the snare wire during such maneuvering, a patient's exposure to x-ray radiation is unduly prolonged.

Another known device used for grasping the distal end of a guidewire and pulling it through a patient's vasculature is a helical snare. The helical snare may be made from a hollow tubular member with a filament wire wrapped around the exterior of the distal end of the tubular member to form a helical snare loop. The filament wire is threaded through the tubular member to emerge at the proximal end of the member. The filament end of the wire may be manipulated to open or close the loop. During surgery, a physician positions the helical snare loop over the distal end of the guidewire and tightens the loop to grasp the guidewire distal end. The guidewire distal end thus lies snug along the body of the hollow tubular member and not transverse to the axis of the tubular member. This reduces the potential for traumatizing the blood vessel walls during withdrawal through the vasculature; however, like the distal loop or snare system, positioning the helical snare is a time consuming process requiring considerable maneuvering and manipulation. Moreover, fluoroscopy is necessary to visualize the maneuvering of the helical snare. Thus, a patient may be exposed to x-ray radiation for a prolonged period of time.

What is needed and heretofore unavailable is a system and method that overcomes the disadvantages of the prior art to position medical devices within a body passage from a distant site, therefore easing the insertion process and facilitating proper device position without the use of advanced training or sophisticated visualization equipment.

### SUMMARY OF THE INVENTION

The present invention inserts two separate guidewires through opposite sites of a body passage and uses magnets on the guidewire distal ends to facilitate a strong direct connection between the distal ends of the guidewires. An access guidewire is inserted into a first insertion site, and a pulling guidewire is inserted into a second insertion site. The distal ends of the two guidewires are positioned proximate to each other so that the magnets will attract each other, thereby connecting the distal ends of the two guidewires. Fluoroscopy or angiography is not required to position the guidewires, but may be useful in positioning and coupling the guidewires. Once connected, the pulling guidewire can then be pulled out of the first insertion site by removing the access guidewire through the first access site. The pulling guidewire is then detached from the access guidewire and attached to a medical device. The pulling guidewire can then be pulled from the second insertion site to guide the medical device into proper placement in the body passage.

The access and pulling guidewires used may be configured from standard medical device guidewires that have one or more magnets mechanically attached to the distal end. The magnet on the access guidewire may incorporate a retaining mechanism to house the magnet on the pulling guidewire and to increase the resistance needed to disconnect the two guidewires. The magnet on the pulling guidewire distal end is configured to easily couple with the magnet on the access guidewire distal end.

A medical device for use with the guidewires of the present invention may be configured with a distal connector that provides for attaching and detaching the pulling guidewire. The connector may be a cam lock design, wherein a portion of the connector is hollowed and shaped to accept the magnet on the pulling guidewire. In such configuration, the magnet on the pulling guidewire locks into the medical device connector and is fixed under tension during insertion of the medical device into the body passage. After the medical device is properly placed, a simple release in tension and twisting action of the pulling guidewire will remove the distal end of the guidewire from the medical device connector.

Alternatively, the medical device connector may incorporate a threaded receptacle. Accordingly, the distal end of the pulling guidewire is configured with a screwhead to accommodate either the magnet or the medical device connector. Other suitable mechanisms, now known or to be developed, for attaching the distal end of the pulling guidewire to the medical device may be employed with the present invention, e.g., ball-and-socket joints and Luer locks. Once the pulling guidewire is pulled out of the first insertion site, the magnetic section may be removed and the screwhead on the pulling guidewire is attached into the threaded connector of the medical device. After insertion of the medical device into the body passage, the pulling guidewire can be rotated to release the connector from the guidewire. The guidewire is then removed from the second access site, leaving the medical device in the desired position in the patient's vasculature.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a schematic representation of two guidewires of the present invention attached to one another via magnets and positioned within a patient's vasculature.

FIG. 2 is schematic representation of two guidewires of the present invention being inserted into a body passage from opposite ends of a patient's vasculature.

FIG. 3 is a schematic representation of two guidewires of the present invention attached at their distal ends positioned within a patient's vasculature.

FIG. 4 is a schematic representation of a pulling guidewire of the present invention being removed through a first insertion site by an access guidewire.

FIG. 5 is a schematic representation of two guidewires of the present invention being detached from one another.

FIG. 6 is a schematic representation of a medical device being attached to a pulling guidewire of the present invention.

FIG. 7 is a schematic representation of a medical device being inserted into a patient's vasculature in accordance with the present invention.

FIG. 8 is a schematic representation of a pulling guidewire of the present invention being detached from a medical device once the medical device has been positioned at a desired location in a patient's vasculature.

FIG. 9 is a schematic representation of a pulling guidewire of the present invention being removed from a patient's vasculature.

FIG. 10 is a perspective view of a first magnet on an access guidewire and a second magnet on a pulling guidewire, the first magnet having a circular lip in accordance with an embodiment of the present invention.

FIG. 11 is a perspective view of a medical device connector with a cam lock design and of a magnet on the distal end of a pulling guidewire of the present invention.

FIG. 12 is a perspective view of a detachable magnet assembly having a threaded guidewire connector, a medical device connector having a threaded distal end, and a pulling guidewire having a screwhead, each in accordance with the present invention.

FIG. 13 is a perspective view of a guidewire with a flexible distal portion.

FIG. 14 is a perspective view of a detachable magnet assembly having a flexible portion and threaded guidewire connector, and a pulling guidewire having a screwhead, each in accordance with the present invention.

### DETAILED DESCRIPTION OF THE PREFERRED EMBODIMENTS

The present invention provides an apparatus and a method for advancing and placing a medical device into the vasculature of a patient that does not require the use of sophisticated visual equipment or the need for advanced training by the physician.

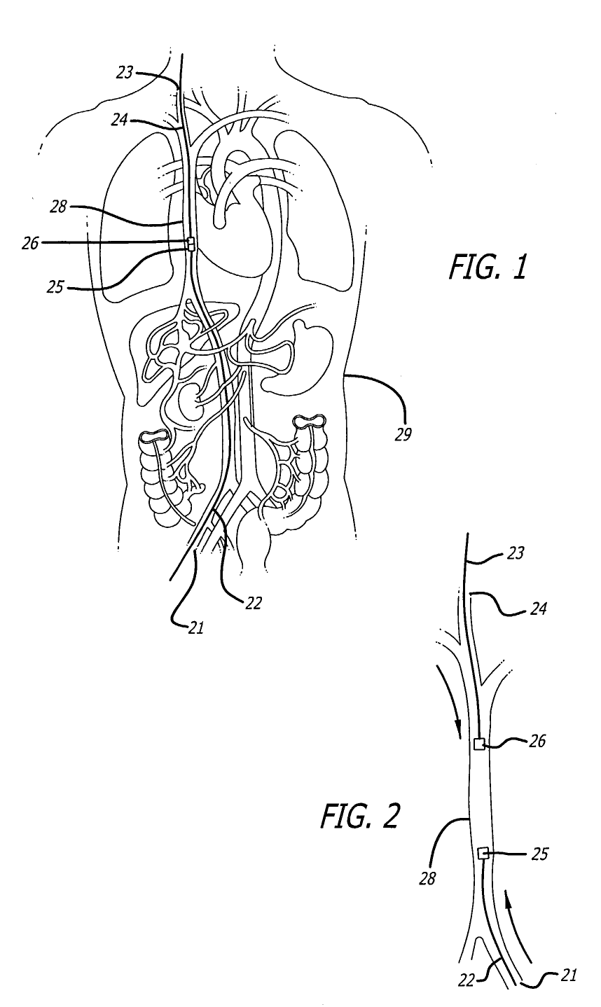

As shown in FIG. 1, an access guidewire 22 and a pulling guidewire 23 may be coupled together via a first magnet 25 and a second magnet 26 within a body passage 28 of a patient 29. The guidewires of the present invention may be standard medical guidewires (for example one mm×260 cm long flexible guidewires). The first magnet 25 may be mechanically attached to a distal end of the access guidewire 22. The second magnet 26 may be mechanically attached to a distal end of the pulling guidewire 23. The magnets may be configured from neodymium-iron-boron (NdFeB) or samarium-cobalt (SmCo) magnets, typically referred to as rare earth magnets.

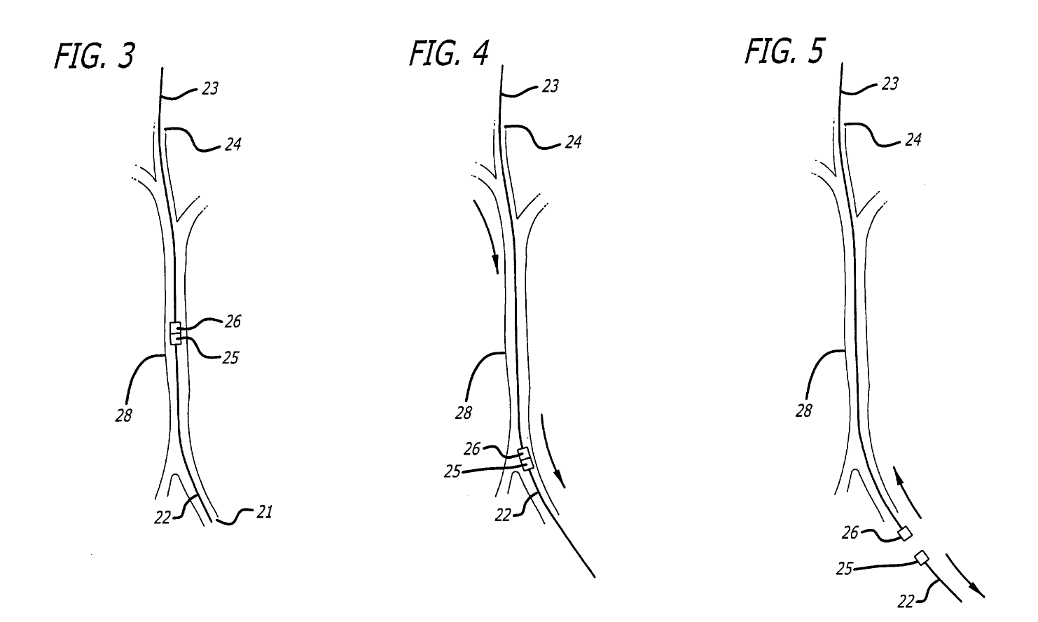

As depicted in FIGS. 2 and 3, the access guidewire 22 is inserted into a first insertion site 21 and advanced along a patient's vasculature (body passage) 28. Likewise, the pulling guidewire 23 is inserted into a second insertion site 24 and advanced along the body passage. As the distal ends of the guidewires near each other, the attached magnets will attract one another and magnetically connect together. An advantage to coupling the guidewires in this way is that the wires can be connected blindly without the use of sophisticated imaging techniques, such as fluoroscopy or angiography. However, such imaging techniques may be useful to assist insertion, placement and coupling of the guidewires and medical device. Once the magnets are coupled, a strong bond is formed such that advancing or retracting either wire from either insertion site will not decouple the magnets. Thus, as shown in FIG. 4, the pulling guidewire 23 can be removed through the first insertion site 21 by pulling the access guidewire 22 out of the body passage 28.

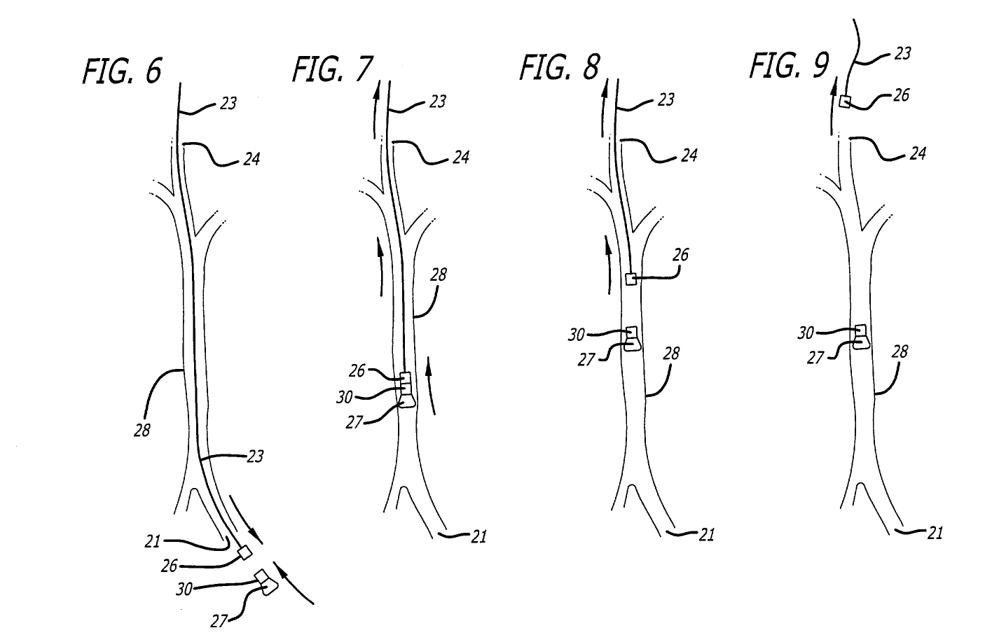

As shown in FIG. 5, the first magnet 25 is detached from the second magnet 26 when the access guidewire 22 is completely out of the body passage 28 and at least the distal end of the pulling guidewire 23 is out of the body passage 28 through the first insertion site 21. Thereafter, the pulling guidewire 23 may be attached to a medical device 27. Upon attachment, the proximal end of the pulling guidewire 23 is manipulated from the outside of the second insertion site 24 (FIG. 6). Pulling on the guidewire proximal end retracts the distal end of the pulling guidewire 23 back into the body passage 28, causing the attached medical device 27 to be advanced through the body passage 28, as shown in FIG. 7.

Once the medical device 27 is positioned at a desired location in the body passage 28, the distal end of the pulling guidewire 23 is detached from the medical device 27, as shown in FIG. 8. The pulling guidewire 23 is then removed completely from the body passage 28 through the second insertion site 24, leaving the medical device 27 in the body passage 28, as shown in FIG. 9.

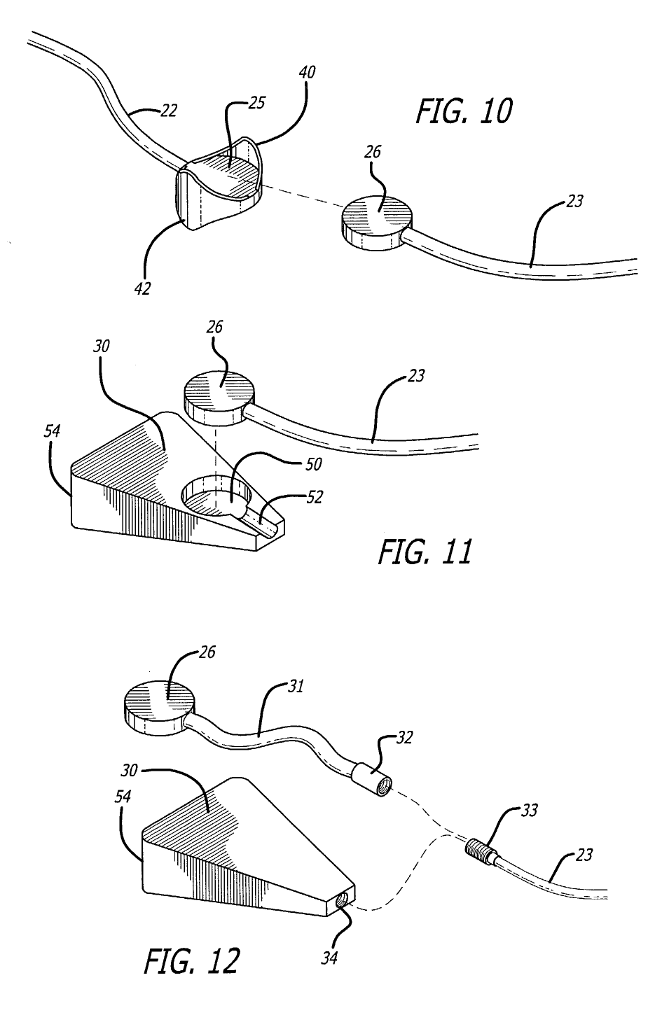

As depicted in FIG. 10, the first magnet 25 may include one or more semi-circular or other suitable shaped flanges or lips 40, 42. The flanges 40, 42 may be sized so that each encloses the circumference of the second magnet 26 when the first magnet 25 and the second magnet 26 attach to one another in the body passage 28. The flanges 40, 42 facilitate retention of the distal ends of the guidewires since the connector 40 adds mechanical resistance by increasing the force necessary to disconnect the magnets from one another. The second magnet 26 lacks such a flange in order to easily couple with the first magnet 25. The flanges 40, 42 may be configured from stainless steel or other suitable biocompatible materials. Although the flanges 40, 42 have been described and shown herein as configured on the first magnet 25, the flanges 40, 42 could similarly be configured on the second magnet 26, thereby achieving the same result. The flanges 40, 42 may be integrally formed with the magnets 25 or 26, or may be formed as a separate structure that encases or is otherwise attached to the magnet 25 or 26.

In one embodiment of the present invention shown in FIG. 11, the medical device 27 includes a connector 30 for removably attaching to the distal end of the pulling guidewire 23. The proximal end 54 of the connector 30 is configured for removably attaching the connector 30 to a medical device 27. The medical device connector 30 may include a cam-lock mechanism at its distal end, wherein a receptacle portion 50 of the connector 30 is formed in a circular or other suitable shape to accept the second magnet 26. The receptacle portion 50 of the connector may taper into a narrow channel 52, enabling the connector to enclose the second magnet 26 in the hollowed portion. The channel 52 is further configured for removably receiving the diameter of the pulling guidewire 23. Such a design allows the second magnet 26 to directly lock into the medical device connector 30 by placing the second magnet 26 into the receptacle portion 50 while allowing the pulling guidewire 23 reside in the narrow channel 52. The second magnet 26 will be fixed under tension during insertion of the medical device 27 into the body passage 28 because the narrow channel 52 will prevent the second magnet 26 from dislodging from the connector 30 when the second magnet 26 lies flat in the receptacle portion 50. After placement of the medical device 27 at a desired location in the body passage 28, a release in tension and twisting action of the pulling guidewire 23 will remove the second magnet 26 from the receptacle portion 50 of the medical device connector 30.

In an alternative embodiment, shown in FIG. 12, the medical device connector 30 may include a threaded lumen 34 at its distal end. The pulling guidewire 23 may be correspondingly configured with a screwhead 33 for engaging a detachable magnet assembly 31 having a threaded guidewire connector 32 or engaging the medical device tip 30 within the threaded lumen 34. Once the pulling guidewire 23 is removed from the first insertion site 21 as shown in FIG. 5, the second magnet 26 may be removed by detaching the detachable magnet assembly 31 from the pulling guidewire 23. The screwhead 33 is then screwed into the threaded lumen 34 of the medical device connector 30. After placement of the medical device 27 at the desired location in the body passage 28, the pulling guidewire 23 can be unscrewed and removed from the medical device connector 30. In addition, alternative mechanisms, known or to be developed, may be used to secure the distal end of the pulling guidewire 23 to the medical device 27, e.g., a ball-and-socket connector, a Luer lock and the like.

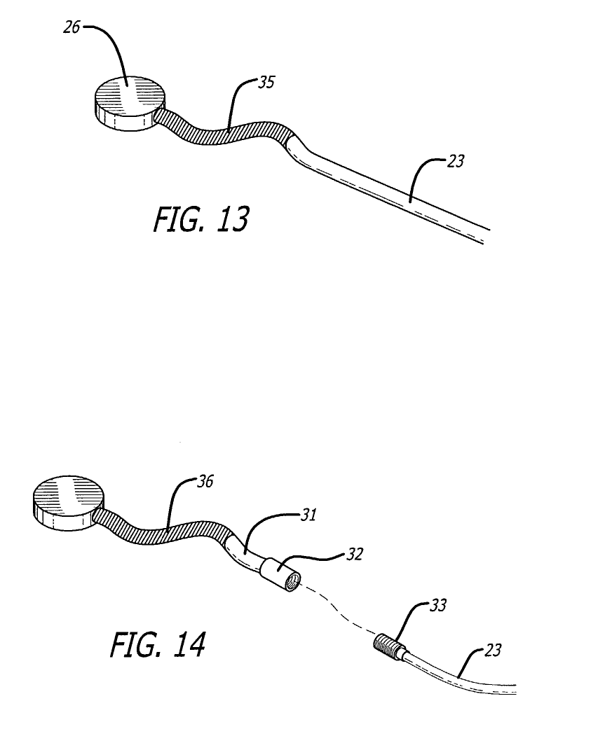

As shown in FIG. 13, either the access guidewire 22 or the pulling guidewire 23 may include a flexible portion 35 to increase the maneuverability of the guidewires in the body passage 28. As shown in FIG. 14, the pulling guidewire 23 may be designed with a screwhead 33. Accordingly, the detachable magnet assembly 31 may include a flexible portion 36 to increase maneuverability in the body passage 28. The flexible portions 35, 36 of the guidewires 22, 23 facilitate the magnetic fields being able to pull the magnets 25, 26 towards one another.

While particular forms of the invention have been illustrated and described, it will also be apparent to those skilled in the art that various modifications and improvements can be made without departing from the spirit and scope of the invention. More specifically, it should be clear that the present invention is not limited to the medical devices described herein, and may be used in conjunction with other types of medical devices. Similarly, a variety of different mechanical mechanisms may be exploited to attach the distal end of the pulling guidewire to the medical device. Accordingly, it is not intended that the invention be limited, except as by the appended claims.

What is claimed is:

1. A system for the introduction and placement of a medical device in a body passage, comprising:
an access guidewire having a distal end for inserting into a first insertion site of the body passage;
a pulling guidewire having a distal end for inserting into a second insertion site of the body passage;
a first magnet attached to the distal end of the access guidewire; and
a second magnet attached to the distal end of the pulling guidewire.

2. The system of claim 1, wherein the first magnet includes a flange for retaining the second magnet.

3. The system of claim 1, further including a connector having a proximal end configured for attaching to a medical device and a distal end configured for attaching to the distal end of the pulling guidewire.

4. The system of claim 3, wherein the medical device connector includes a cam lock for attaching the second magnet on the pulling guidewire.

5. The system of claim 3, wherein the medical device connector includes a threaded lumen for removably attaching to a screwhead on the pulling guidewire.

6. The system of claim 1, wherein at least one of the access guidewire and the pulling guidewire includes a flexible portion.

7. A method for the introduction and placement of a medical device in a vasculature of a patient, comprising:
providing a first guidewire having a distal end attached to a first magnet;
providing a second guidewire having a distal end attached to a second magnet;
inserting the first guidewire into a first insertion site of a patient vasculature;
inserting the second guidewire into a second insertion site of the patient vasculature;
positioning the first magnet proximal to the second magnet to removably couple the distal end of the first guidewire to the distal end of the second guidewire;
manipulating the first guidewire to remove the second guidewire from the first insertion site;
uncoupling the first guidewire from the second guidewire;
attaching the distal end of the second guidewire to a medical device; and
retracting the second guidewire through the second insertion site, so as to position the medical device at a desired location in the patient vasculature.

8. The method of claim 7, further comprising configuring the first magnet with means for mechanically retaining the second magnet.

9. The method of claim 7, further comprising configuring the medical device with a connector for removably attaching the medical device to the second guidewire.

10. The method of claim 9, further comprising configuring the medical device connector with a receptacle for removably receiving the second magnet.

11. The method of claim 9, further comprising configuring the medical device connector with a threaded lumen for removably attaching to a screwhead on a distal portion of the second guidewire.

12. The method of claim 7, further comprising configuring the first guidewire with a flexible portion proximate the distal end of the first guidewire.

13. The method of claim 7, further comprising configuring the second guidewire with a flexible portion proximate the distal end of the second guidewire.
